(19) United States
(12) Patent Application Publication
(10) Pub. No.: US 2009/0194103 A1
(43) Pub. Date: Aug. 6, 2009

(54) ANESTHESIA DEVICE WITH STAND-ALONE VAPORIZER APPARATUS

(75) Inventors: Mark Alan Thom, Madison, WI (US); Robert Tham, Middleton, WI (US); John Raymond Pinkert, Madison, WI (US); Andrew Jungwirth, Madison, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/023,118

(22) Filed: Jan. 31, 2008

(51) Int. Cl. A61M 16/01

(52) U.S. Cl. 128/203.12

(57) ABSTRACT

An anesthesia system is disclosed herein. The system includes an anesthesia machine comprising an anesthesia machine input, and a stand-alone vaporizer connected to the anesthesia machine. The stand-alone vaporizer includes a vaporizer input. The stand-alone vaporizer is configured to produce a selectable concentration of an anesthetic agent. The anesthesia machine input and the vaporizer input may be independently implemented to regulate the concentration of the anesthetic agent from the stand-alone vaporizer.

### BACKGROUND OF THE INVENTION

The subject matter disclosed herein relates to a system comprising an anesthesia device and a stand-alone vaporizer.

Conventional anesthesia delivery systems include an anesthesia machine pneumatically coupled with a remotely located stand-alone vaporizer. The anesthesia machine generally receives a plurality of different gasses such as air, O2 and/or N20 from a wall outlet and combines them in a selectable manner. The stand-alone vaporizer introduces a selectable concentration of vaporized anesthetic agent into the gas mixture. The vaporized anesthetic agent and gas mixture are then transferred to a patient.

One problem with some conventional anesthesia delivery systems is that the anesthesia machine and the vaporizer generally must be controlled via separate interfaces. As an example, an operator must generally set the individual concentrations of the air, O2 and N20 using a first interface disposed on the anesthesia machine, and must thereafter set the concentration of the anesthetic agent using a second interface disposed on the remotely located stand-alone vaporizer. It can be seen that it is potentially less efficient to operate a control system comprising two disparate and remotely located user interfaces as compared to a control system implementing a single interface.

### BRIEF DESCRIPTION OF THE INVENTION

The above-mentioned shortcomings, disadvantages and problems are addressed herein which will be understood by reading and understanding the following specification.

In an embodiment, an anesthesia machine includes a controller, a motor connected to the controller, and a coupling operatively connected to the motor and adapted for engagement with a stand-alone vaporizer. The coupling is configured to transmit energy from the motor to the stand-alone vaporizer in order to regulate a concentration of an anesthetic agent.

In another embodiment, a system includes an anesthesia machine comprising an anesthesia machine input, and a stand-alone vaporizer connected to the anesthesia machine. The stand-alone vaporizer includes a vaporizer input. The stand-alone vaporizer is configured to produce a selectable concentration of an anesthetic agent. The anesthesia machine input and the vaporizer input may be independently implemented to regulate the concentration of the anesthetic agent from the stand-alone vaporizer.

In another embodiment, a system includes an anesthesia machine comprising a controller and a motor connected to the controller. The system also includes a stand-alone vaporizer connected to the anesthesia machine. The stand-alone vaporizer including a vaporizer input. The system also includes a coupling disposed between the motor and the vaporizer input. The coupling is configured to transmit energy from the motor to the vaporizer input in order to regulate the concentration of an anesthetic agent. The system also includes a connection disposed between the controller and the stand-alone vaporizer. The connection is configured to transmit data from the stand-alone vaporizer to the controller.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating an anesthesia system including a stand-alone vaporizer in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

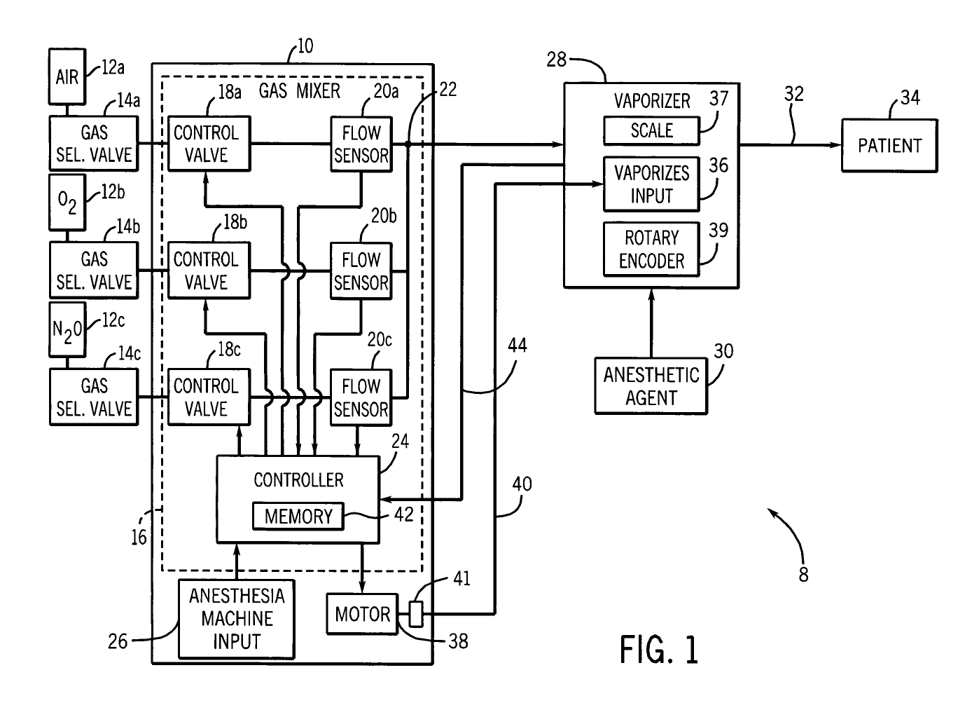

Referring to FIG. 1, an anesthesia system 8 is schematically depicted in accordance with one embodiment. The anesthesia system 8 includes an anesthesia machine 10, a plurality of gas storage devices 12a, 12b and 12c, and a stand-alone vaporizer 28. The anesthesia machine 10 is shown for illustrative purposes and it should be appreciated that other types of anesthesia machines may alternately be implemented. In a typical hospital environment, the gas storage devices 12a, 12b and 12c each comprise a centrally located storage tank configured to supply medical gas to multiple hospital rooms via a wall outlet. The storage tanks are generally pressurized to facilitate the transfer of the medical gas to the anesthesia machine 10.

The gas storage devices 12a, 12b and 12c will hereinafter be described as comprising an air tank 12a, an oxygen (O2) tank 12b, and a nitrous oxide (N20) tank 12c, respectively, however it should be appreciated that other storage devices and other types of gas may alternatively be implemented. The gas storage tanks 12a, 12b and 12c are each connected to one of the gas selector valves 14a, 14b, and 14c. The gas selector valves 14a, 14b and 14c may be implemented to shut off the flow of medical gas from the storage tanks 12a, 12b and 12c when the anesthesia machine 10 is not operational. When one of the gas selector valves 14a, 14b and 14c is opened, gas from a respective storage tank 12a, 12b and 12c is transferred under pressure to the anesthesia machine 10.

The anesthesia machine 10 includes a gas mixer 16 adapted to receive medical gas from the storage tanks 12a, 12b and 12c. The gas mixer 16 includes a plurality of control valves 18a, 18b and 18c that are respectively connected to one of the gas selector valves 14a, 14b and 14c. The gas mixer 16 also includes a plurality of flow sensors 20a, 20b and 20c that are each disposed downstream from a respective control valve 18a, 18b, and 18c. After passing through one of the control valves 18a, 18b and 18c, and passing by one of the flow sensors 20a, 20b and 20c, the air, O2 and N20 are combined to form a mixed gas at the mixed gas outlet 22.

The control valves 18a, 18b and 18c and the flow sensors 20a, 20b and 20c are each connected to a controller 24. The controller 24 is configured to operate the control valves 18a, 18b and 18c in response to user input from the anesthesia machine input 26, and gas flow rate feedback from the sensors 20a, 20b and 20c. The anesthesia machine input 26 may comprise any known input device such as, for example, a touch screen, keyboard, mouse, joystick, etc. According to one embodiment, a user can specify air, O2 and N20 concentrations via the anesthesia machine input 26, and thereafter the controller 24 regulates the control valves 18a, 18b and 18c in a manner adapted to produce the user specified concentrations of air, O2 and N20 at the mixed gas outlet 22. The controller may additionally be configured to adjust the control valves 18a, 18b and 18c in response to feedback from the sensors 20a, 20b and 20c if, for example, the measured concentrations of the air, O2 and N20 are inconsistent with the user specified concentrations.

The mixed gas from the mixed gas outlet 22 is transferred to the stand-alone vaporizer 28. For purposes of this disclosure, a stand-alone vaporizer is a discrete component of an anesthesia system disposed separately from the anesthesia machine 10. As the stand-alone vaporizer 28 is a discrete component of the system 8, it is more accessible and therefore easier to service, repair and/or replace as compared to an integral vaporizer incorporated into the design of an anesthesia machine. The stand-alone vaporizer 28 is configured to vaporize an anesthetic agent 30, and to combine the vaporized anesthetic agent with the mixed gas from the mixed gas outlet 22. The vaporized anesthetic agent and mixed gas combination passes through a breathing tube 32 and is delivered to the patient 34.

The stand-alone vaporizer 28 includes a vaporizer input 36 adapted to allow a user to regulate the concentration of vaporized anesthetic agent transferred to the patient. The vaporizer input 36 will hereinafter be described as a concentration dial for illustrative purposes; however, other input devices may be envisioned. The concentration dial 36 is a manual device comprising a rotary type dial that is adapted to regulate vaporized anesthesia agent concentration based on the degree of dial rotation. According to one embodiment, the stand-alone vaporizer 28 includes a scale 37 disposed about the periphery of the of the concentration dial 36. The scale 37 comprises a system of ordered marks positioned at fixed intervals relative to the concentration dial 36 so that the degree of dial rotation is visually identifiable. According to another embodiment, the stand-alone vaporizer 28 includes a rotary encoder 39 or similar device adapted to identify the rotational position of the concentration dial 36.

The anesthesia system 8 is configured such that a user can regulate the concentration of vaporized anesthesia agent using the vaporizer input 36 in the manner previously described, or using the anesthesia machine input 26 as will be described in detail hereinafter. The ability to use either of two different input devices (i.e., the anesthesia machine input 26 or the vaporizer input 36) to operate the stand-alone vaporizer 28 increases the likelihood that the vaporizer 28 will remain operational by providing an input device backup. As an example, if a power shortage renders the anesthesia machine input 26 inoperable, a vaporizer input device such as the previously described manual concentration dial can be implemented to operate the stand-alone vaporizer 28. The ability to operate the stand-alone vaporizer 28 via the anesthesia machine input 26 also allows a user to operate two devices (i.e., the anesthesia machine 10 and the vaporizer 28) using a single interface. It can be seen that operating the anesthesia machine 10 and the stand-alone vaporizer 28 via a single interface can improve efficiency as compared to a system wherein a user must set the individual concentrations of air, O2 and N20 using a first interface disposed on the anesthesia machine, and then set the concentration of vaporized anesthetic agent using a second interface disposed on a remotely located vaporizer.

According to one embodiment, the anesthesia machine input 26 is adapted to regulate vaporized anesthesia agent concentration via a motor 38 and a coupling 40. According to the depicted embodiment, the motor 38 is a component of the anesthesia machine 10; however, it should be appreciated that the motor 38 may alternatively be integrated into the design of the stand-alone vaporizer 28 or may be independently disposed. Similarly, the coupling 40 may comprise a component of the anesthesia machine 10, the stand-alone vaporizer 28, or may be independently disposed. In a non-limiting manner, the coupling 40 may comprise a mechanical device; a pneumatic device, a magnetic device, and/or an electronic device. For illustrative purposes, the coupling 40 will hereinafter be described as a drive shaft that mechanically couples the motor 38 with the vaporizer input 36.

The motor 38 is operatively connected to the controller 24 and the drive shaft 40. The motor 38 may be operated by the controller 24 in response to a user command from the anesthesia machine input 26. According to the depicted embodiment, the controller 24 includes a memory device 42 containing calibration data. Alternatively, the memory device 42 may be comprise a component of the anesthesia machine 10 disposed remotely relative to the controller 24, or may comprise a component of the stand-alone vaporizer 28.

Calibration data from the memory device 42 may comprise a table or graph correlating the rotational position of the concentration dial 36 with anesthetic agent concentration. The calibration data can be implemented to identify a target rotational position of the concentration dial 36 based on a user specified anesthetic agent concentration. The controller 24 can develop an appropriate motor command based on the current position of the concentration dial 36, which is obtainable from the rotary encoder 39, and the target rotational position of the concentration dial 36. The motor command may, for example, specify the direction in which the motor is operated (i.e., forward or reverse), the motor speed, the angular position of the motor, and/or the duration of motor operation. The controller 24 can operate the motor 38 in accordance with one or more motor commands developed in the manner previously described in order to deliver the user specified anesthetic agent concentration.

Output from the motor 38 may be transmitted via the drive shaft 40 to the vaporizer input 36 in a manner that physically translates and/or rotates the vaporizer input 36 relative to the scale 37. Accordingly, a user can implement the anesthesia machine input 26 to select a given concentration of vaporized anesthetic agent at the stand-alone vaporizer 28 without directly engaging the vaporizer 28. Additionally, by physically translating and/or rotating the vaporizer input 36 in the manner described, the anesthetic agent concentration being delivered to the patient 34 is visually identifiable.

The anesthesia system 8 may optionally include a motor disengagement device 41. Although depicted as a component of the anesthesia machine 10, the disengagement device 41 also may be included as a component of the vaporizer 28 or as an independent component. The disengagement device 41 may, for example, comprise a quick-release feature adapted to physically decouple the motor 38 from the driveshaft 40, or a one-way clutch adapted to interrupt the transfer of torque from the vaporizer input 36 to the motor 38. In the absence of the optional disengagement device 41, direct manual actuation of the vaporizer input 36 could impart a force tending to back-drive the motor 38. The process of back-driving the motor 38 introduces unnecessary resistance that can impede vaporizer input 36 actuation. Accordingly, the disengagement device 41 may be implemented to selectively decouple the motor 38 from the stand-alone vaporizer 28 and to thereby minimize resistance associated with the regulation of anesthetic agent concentration via the vaporizer input 36. The disengagement device 41 may also comprise a device adapted to deactivate or de-energize the motor 38 as an alternative approach to minimizing resistance.

According to one embodiment, the anesthesia system 8 includes a connection 44 adapted to transmit data from the stand-alone vaporizer 28 to the controller 24. The connection 44 may comprise a component of the anesthesia machine 10, the stand-alone vaporizer 28, or may be independently disposed. The connection 44 may, for example, comprise an electrical connection; a wireless connection; an optical connection; or any other known connection through which data is transferable. Although the connection 44 and the coupling 40 are schematically shown as separate devices, they may alternatively be combined into a single component.

The connection 44 may be implemented to transmit a variety of different types of data or information. The following will provide some non-limiting examples of types of data that may be transmitted from the stand-alone vaporizer 28 to the controller 24 via the connection 44. According to one embodiment, the transmittable data may comprise information indicating that the vaporizer 28 has been properly installed or connected to the anesthesia machine 10. According to another embodiment, the transmittable data may comprise information identifying the specific type of anesthetic agent being introduced to the patient 34 by the vaporizer 28. According to another embodiment, the transmittable data may comprise calibration information correlating anesthetic agent concentration with the position of the vaporizer input 36 (e.g., the degree to which the vaporizer input 36 is translated and/or rotated). According to another embodiment, the transmittable data may comprise information pertaining to the temperature and/or pressure of the anesthetic agent 30 in the stand-alone vaporizer 28. According to another embodiment, the transmittable data may comprise information identifying the current translational and/or rotational position of the vaporizer input 36 that may be obtainable from the rotary encoder 39. Such data may be useful as a safety check for the proper operation of the anesthetic agent delivery system.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An anesthesia machine comprising:
a controller;
a motor connected to the controller; and
a coupling operatively connected to the motor and adapted for engagement with a stand-alone vaporizer, said coupling being configured to transmit energy from the motor to the stand-alone vaporizer in order to regulate a concentration of an anesthetic agent.

2. The anesthesia machine of claim 1, further comprising a memory device connected to the controller, said memory device being adapted to retain calibration data.

3. The anesthesia machine of claim 1, further comprising a connection disposed between the controller and the stand-alone vaporizer, said connection being configured to transmit data from the stand-alone vaporizer to the controller.

4. The anesthesia machine of claim 3, wherein the connection is configured to transmit calibration data.

5. The anesthesia machine of claim 3, wherein the connection is configured to transmit data pertaining to an anesthetic agent type, an anesthetic agent temperature, a position of a stand-alone vaporizer input device, and/or an anesthetic agent pressure.

6. The anesthesia machine of claim 3, wherein the connection is selected from the group consisting of an electrical connection; a wireless connection; and an optical connection.

7. The anesthesia machine of claim 1, wherein the coupling is selected from the group consisting of a mechanical coupling, a pneumatic coupling, a magnetic coupling, and an electronic coupling

8. The anesthesia machine of claim 1, further comprising a device configured to selectively decouple the motor from the stand-alone vaporizer.

9. The anesthesia machine of claim 1, further comprising a device configured to selectively deactivate the motor.

10. A system comprising:
an anesthesia machine including an anesthesia machine input; and
a stand-alone vaporizer connected to the anesthesia machine, said stand-alone vaporizer including a vaporizer input, said stand-alone vaporizer being configured to produce a selectable concentration of an anesthetic agent;
wherein the anesthesia machine input and the vaporizer input may be independently implemented to regulate the concentration of the anesthetic agent from the stand-alone vaporizer.

11. The system of claim 10, further comprising a motor operatively connected to the vaporizer input, said motor being adapted to physically translate and/or rotate the vaporizer input.

12. The system of claim 11, further comprising a coupling disposed between the motor and the vaporizer input.

13. The system of claim 12, wherein the coupling comprises a drive shaft.

14. The system of claim 10, further comprising a connection disposed between the anesthesia machine and the stand-alone vaporizer, said connection being configured to transmit data.

15. The system of claim 15, wherein one of the anesthesia machine and the stand-alone vaporizer includes a memory adapted to retain calibration data.

16. The system of claim 10, further comprising a device configured to selectively deactivate the motor and/or to selectively decouple the motor from the stand-alone vaporizer.

17. A system comprising:
an anesthesia machine including:
a controller; and
a motor connected to the controller;
a stand-alone vaporizer connected to the anesthesia machine, said stand-alone vaporizer including a vaporizer input;
a coupling disposed between the motor and the vaporizer input, said coupling being configured to transmit energy from the motor to the vaporizer input in order to regulate a concentration of an anesthetic agent; and
a connection disposed between the controller and the sand-alone vaporizer, said connection being configured to transmit data from the stand-alone vaporizer to the controller.

18. The system of claim 17, wherein one of the anesthesia machine and the stand-alone vaporizer includes a memory adapted to retain calibration data

19. The system of claim 17, further comprising a device configured to selectively deactivate the motor and/or to selectively decouple the motor from the stand-alone vaporizer.

20. The system of claim 17, wherein the stand-alone vaporizer includes a scale disposed in close proximity to the vaporizer input.
